(19) United States
(12) Patent Application Publication
(10) Pub. No.: US 2016/0367194 A1
(43) Pub. Date: Dec. 22, 2016

(54) ILLUMINATION SYSTEM FOR MOBILE DEVICES

(71) Applicant: Richard Murphy, San Diego, CA (US)

(72) Inventor: Richard Murphy, San Diego, CA (US)

(21) Appl. No.: 15/156,324

(22) Filed: May 16, 2016

Related U.S. Application Data
(60) Provisional application No. 62/161,868, filed on May 14, 2015.

(51) Int. Cl. A61B 5/00; H04N 7/14; A61N 5/06; G06F 19/00; A61B 5/01; H04N 5/225; H04N 5/33

(52) U.S. Cl. CPC A61B 5/6898; H04N 5/2256; H04N 7/147; H04N 5/33; G06F 19/3418; G06F 19/345; A61B 5/0008; A61B 5/0013; A61B 5/0022; A61B 5/0075; A61B 5/0077; A61B 5/015; A61B 5/411; A61B 5/444; A61B 5/445; A61B 5/447; A61B 5/4836; A61N 5/0616

(57) ABSTRACT

The present invention provides an apparatus for reversible stable association with a portable computing and communications device (PCCD) and illuminate the proximity resulting in illumination of an object in the proximity. The present invention provides a compact portable system to facilitate enhanced imaging of an individual employing a portable computing and communications device (PCCD) to participate in a videoconference. The present invention provides an apparatus that retains the PCCD device and provides controlled and customizable illumination of the videoconference participant which results in enhanced imaging of the videoconference participant. The apparatus of the present invention is useful in the practice of telemedicine.

### CROSS REFERENCE TO OTHER APPLICATIONS

This application is related to and claims the benefit pursuant to 35 U.S.C. §119 of U.S. Provisional Patent Application Ser. No. 62/161,868 filed May 14, 2015.

### BACKGROUND OF THE INVENTION

The term “videotelephony” describes the simultaneous transmission of audio-visual signals by users at different locations to provide visual and voice communication between people in real-time over a network. The ability to both see and hear the person in a remote location in real time has been been an alluring prospect since the early days of the telephone and came into practical application with the advent of the television in the late-1920s. Videotelephone systems were in public use in the late 1930s in Germany. AT&T began to produce commercial videotelephony systems, the “Picturephone”, in the 1960s. The 1968 motion picture “2001: A Space Odyssey” predicted a future where videotelephony would be so commonplace as to be used by children. Despite these technological advancements, these videotelephone systems were not commercial successes due primarily to cost and the obstacles to transmitting large quantities of data over existing network systems.

The term “videoconferencing” has replaced the term videotelephony in modern usage. The advent of video compression technologies in the 2000s enabled high quality transmission of video and voice data over computer networks and made the videotelephony technology widely available. Modern portable computing and communication devices (PCCDs) such as smartphones and tablets contain sophisticated camera systems and powerful computer processors capable of enabling videoconferencing in almost any location. The term “videoconferencing software” refers to any of a variety of software application to enable PCCD-based videoconferencing over a network. Examples of videoconferening software useful in the practice of the present invention include Skype™ (Microsoft Corporation, Redmond Wash.) and FaceTime® (Apple, Inc., Cupertino Calif.).

Although the desire to both see and hear others at a distance that has intrigued us since the first videotelephone systems were postulated at the turn of the last century and the advances in hardware and software that has put the technology within the reach of a majority of the population, people have yet to adopt the technology at the rate that would have been expected. According to reports, in 2009 approximately only 34% of the calls conducted over the Skype system were video calls. Studies have examined a variety of factors to attempt to explain why a technology that had been anticipated for more than 70 years and for which the initial technological hurdles have been overcome so as to place the technology in the home (or hand) of a wide section of society has not been as widely adopted as one would have expected. Although privacy has been highlighted as a major concern, a significant impediment to the adoption of this technology is that people simply do not like the way that they appear on screen. Jose Eurico de Vasconcelos Filo, Kori Inkpen, and Mary Czerwinski, “Image, Appearance and Vanity in the Use of Media Spaces and Videoconference Systems,” International ACM conference on Supporting group work (Group 2009), Association for Computing Machinery, Inc., May 2009. The option to work remotely or telecommute has compelled some individuals to interact using these systems for their business. As individuals have been compelled to use the technology as a condition of their employment, some have even resorted to the seemingly drastic option of plastic surgery to improve their appearance on screen introducing the term “FaceTime Facelift” into the popular lexicon. See e.g., “FaceTime Facelift: The Plastic Surgery Procedure For iPhone Users Who Don't Like How They Look On FaceTime”, by Jason Gilbert, The Huffington Post Tech, Feb. 27, 2012 and “Ready for My Video Chat Close-Up” by Austin Considine, NY Times, Apr. 17, 2012.

A primary reason that PCCD users perceive poor image quality in videoconferences conducted using PCCD devices (“PCCD mediated videoconferences”) is the inadequate and/or improper lighting of the user. Ambient lighting is generally unsuitable for proper illumination of the PCCD user to provide a realistic appearance. As a practical matter, in the typical home or work environment there is little that can be done to achieve improved lighting for a video conference. It is difficult for the typical consumer to incur the effort and expense to install auxiliary lighting in the home or workplace so as to provide a realistic image on a videoconference call. Furthermore, PCCD devices are by their nature portable leading to their use in a wide variety of situations rendering a complex independent illumination system impractical.

Similarly, the cameras of most PCCDs typically rely solely on ambient lighting to provide illumination of the PCCD user when taking a still image using the camera (e.g. a self-portrait or “selfie”) or using the video mode for the camera. When creating a self-video, it is more convenient to be able to visualize the scene being captured by the front facing camera using the display as the viewfinder. However, making such videos again primarily relies on the ambient lighting which is frequently suboptimal for proper imaging.

Additionally, the availability of medical care to individuals located in remote locations or who are unable to travel to a medical facility for treatment is significant. This need has given rise to the field of telemedicine, the use of information technologies to provide health care at a distance. However, in order to conduct such remote caregiving, the currently available technologies are reliant on ambient light and are unable to control the lighting at the remote patient location.

Consequently, there is a need in the art for a portable illumination system that provides illumination of a PCCD user employing the front-facing camera of a PCCD.

### SUMMARY OF THE INVENTION

The present invention provides an apparatus to facilitate enhanced imaging of objects in the proximity to the camera of a portable computing and communications device (PCCD). The present invention provides an apparatus that is to be associated with a PCCD device and provides controlled and customizable illumination of the PCCD user resulting in enhanced imaging of the PCCD user for video capture by the camera of a PCCD.

In one embodiment, the apparatus provides a control system in operable communication with the PCCD device providing integration of the illumination system (and other controls such as audio) of the present invention with the videoconferencing software provided on the PCCD device so as to automatically engage the illumination system and other optional features of the apparatus in response to the PCCD receiving a request to participate in a videoconference.

### BRIEF DESCRIPTION OF THE FIGURES

FIG. 1 of the attached drawings provides an exploded lower front perspective view of one embodiment of the apparatus of the present invention illustrating a housing that incorporates an illumination source, diffuser element, and recess to receive a PCCD, providing a means for reversible stable association with said PCCD.

FIG. 2 of the attached drawings provides an exploded upper rear perspective view of one embodiment of the apparatus of the present invention illustrating a housing, the rear surface of which provides for a recess to receive a battery and battery cover and also provides a support means, shown retracted, and a recess to receive the support means when retracted.

FIG. 3 of the attached drawings provides a front perspective exploded view of one embodiment of the apparatus of the present invention illustrating a housing the front face of which provides a recess to receive and stably maintain a PCCD, the front face of the recess providing an additional recess to receive a battery and battery cover. This figure also provides exemplary illustrations of various optional features, such as speaker ports to redirect sound forward, to provide access for charging cords, external switches to control illumination.

FIG. 4 of the attached drawings provides a front right perspective view of one embodiment of the apparatus of the present invention illustrating a housing the front face of which provides a recess to receive and stably maintain a PCCD, the front face of the recess providing an additional recess to receive a battery and battery cover. This figure also provides exemplary illustrations of various optional features, such as speaker ports to redirect sound forward, to provide access for charging cords, external switches to control illumination.

FIG. 5 of the attached drawings provides a top right front perspective view of one embodiment of the apparatus of the present invention illustrating a housing the front face of which provides a recess to receive and stably maintain a PCCD, the front face of the recess providing an additional recess to receive a battery and battery cover. This figure also provides exemplary illustrations of various optional features, such as speaker ports to redirect sound forward, to provide access for charging cords, external switches to control illumination.

FIG. 6 of the attached drawings provides a lower right rear perspective view of one embodiment of the invention illustrating the support provided on the rear face of the housing to maintain the housing at a prescribed angle relative to a support surface.

FIG. 7 of the attached drawings provides an exploded perspective view of one configuration of the apparatus of the present invention wherein the front wall (100F) of the housing (100) is provides a recess (114) to reversibly receive a PCCD-specific insert (115), the front wall (115F) of said PCCD-specific insert (115) providing a recess (117) that conforms substantially to the external dimensions of the PCCD (105) such that when the insert (115) is associated with the housing (100) the PCCD (105) is maintained in stable association with the housing, the dashed arrows indicating the direction of insertion of the PCCD (105), PCCD specific insert (115) relative to the housing (100).

FIG. 8 of the attached drawings provides a block diagram of the electrical components of the apparatus of the present invention. The solid lines indicate an embodiment where electrical communication between components is provided by conventional electrical conductors. The dotted lines are used to indicate operable communication between components by either wired or wireless communication. The dashed lines around the power supply is provided to illustrate that the power supply may be provided integral to the housing or positioned remotely (including a power supply incorporated into the PCCD).

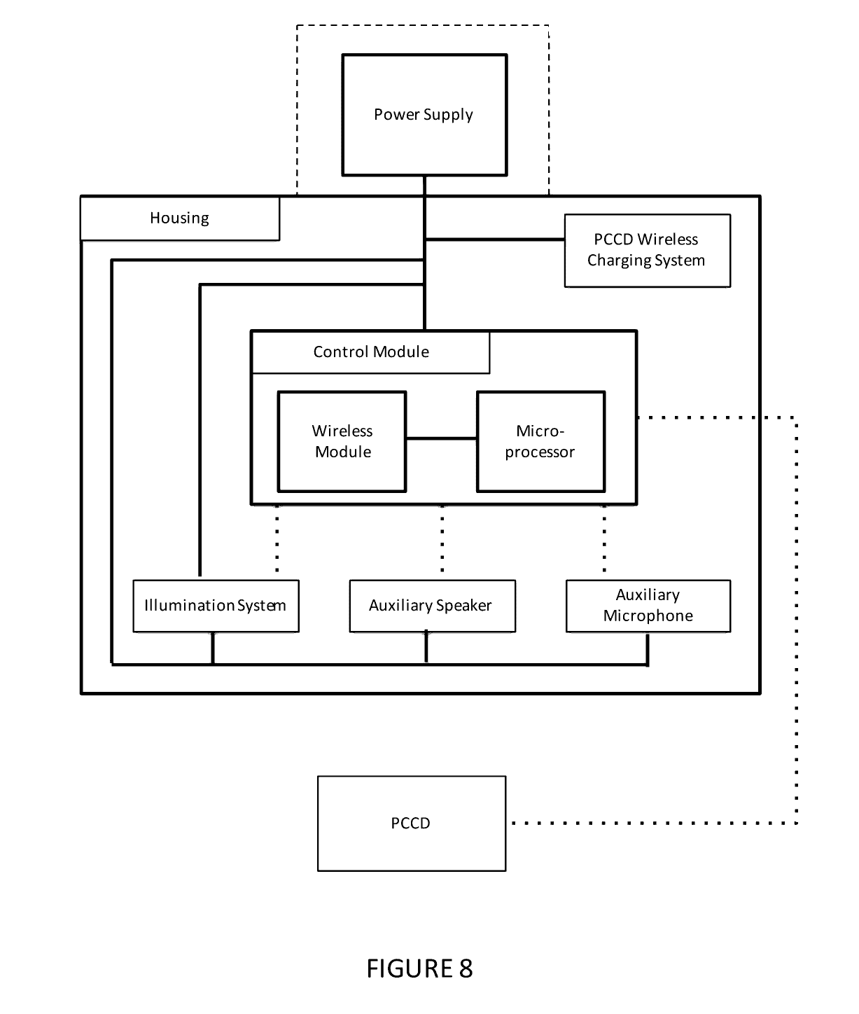

FIG. 9 of the attached drawings provides a perspective view of one embodiment of the invention wherein the apparatus is provided in a housing for stable and reversible association with a PCCD being provided by a protrusion from the surface of the housing which is received by a port of a PCCD device.

FIG. 10 of the attached drawings provides a perspective view of one embodiment of the invention wherein the apparatus is provided in a housing for stable and reversible association with a PCCD being provided by a protrusion from the surface of the housing which is received by a port of a PCCD device, the housing having elongated walls defining a recess to provide enhanced stable and reversible association between the PCCD and housing.

### DETAILED DESCRIPTION OF THE INVENTION

Detailed embodiments of the present invention are disclosed herein. However, it is to be understood that the disclosed embodiments are merely illustrative of the invention that may be embodied in various forms. In addition, each of the examples given in connection with the various embodiments of the invention is intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention. Furthermore, any section headings are merely for convenience of the reader and not intended to provide a limitation on the scope of the disclosure with respect to any feature of utility of the present invention.

As used herein the term “camera” refers to the front-facing camera of a PCCD. The term “front-facing” refers to the situation where the camera aperture and the display of the PCCD are on the same side of the PCCD. Such cameras typically comprise a digital image sensor capable of acquiring and digitizing images and one or more lenses to focus the image on the image sensor. Examples of digital image sensors include but are not limited to the charge coupled device (CCD) or the complementary metal-oxide-semiconductor (CMOS) active pixel sensor imagers.

As used herein the term “display” is used in its conventional sense to refer to a component of a PCCD capable rendering images and text for viewing by a user. Displays may be of various types including but not limited to liquid crystal displays, IPS light emitting diodes (LED), polymer light emitting diodes (PLED), organic light emitting diodes (OLED), polymer organic light emitting diodes (POLED), passive matrix organic light emitting diode (PMOLED), or active matrix organic light emitting diode (AMOLED) arrays. Displays may render computer output but also may also incorporate touch screen technology enabling user input to facilitate manipulation (e.g. zoom, rotation, or perspective) of the image provided on the display in conjunction with software resident on the PCCD.

The term “portable computing and communications device” or “PCCD” as used herein is used to describe a portable electronic device comprising: (a) a camera; (b) a microphone; (c) a means to transmit video and audio information receive from said camera and microphone, respectively, over a network; (d) a means to receive audio and/or video information from a second camera and/or microphone over a network; (f) a means for storing digital information; (g) a means to decode digital visual and audio signals received by the device; (h) a display; and (i) a means to broadcast said decoded audio signal locally (e.g. a speaker). The term PCCDs includes smartphones, tablets, and hybrid (commonly referred to as “phablet”) devices having a front facing camera and display. Examples of PCCDs useful in the practice of the present invention include, but are not limited to, the iPhone® series of smartphones including the iPhone® models 4, 4S, 5, 5S, 5C, 6 and 6 Plus, the iPad® tablet devices including the iPad Air®, iPad Mini®, iPad Pro®, iPod Touch® devices (commercially available from Apple, Inc. Cupertino, Calif.), Lumia® series smartphone and Surface® series tablet devices (commercially available from Microsoft Corporation, Redmond Wash.), Galaxy® series smartphones and tablet devices (Samsung Corporation), Aspire® and Liquid® series smartphones and Iconia® and Predator® series tablet devices (commercially available from Acer, Inc, New Taipei City, Taiwan), ZenPhone® and PadFone® series of smartphones (commercially available from Asustek Computer Inc. Taipei, Taiwan), IdeaPad® series tablets (commercially available from Lenovo Group Ltd, Beijing, China), OnePlus® series smartphones (commercially available from Shenzhen OnePlus Science and Technology Co, Ltc, Shenzhen, Guangdong, China), and Nexus® series smartphone and tablet devices (Google, Inc., Mountain View Calif.).

As used herein the term “PCCD-mediated videoconference” refers to a videoconference in which at least one participant in the videoconference is participating in the videoconference using a PCCD. PCCD mediated videoconferences are generally facilitated by the execution of videoconferencing software resident on the PCCD or a remote computing device in communication with the PCCD. Examples of such videoconferencing software include, but are not limited to, the SkypeTM and FaceTime® software commercially available from Microsoft Corporation and Apple, Inc. respectively. In a typical operation of a PCCD-mediated videoconference, the PCCD user initiates a software application installed on the PCCD device which communicates over a network with a remote computing device resulting in simultaneous transfer of image and voice data over a network.

As used herein the term “proximity” refers to the region in front of a PCCD viewable by the front facing camera within less than about 3 meters, 2 meters or 1 meter from the camera. When the PCCD is associated with the apparatus of the present invention, the illumination source provides illumination of the proximity. Similarly, the term “proximal” is used to refer to a condition of being within the proximity.

As used herein the term “videoconference” is used to generally describe the simultaneous transmission of audio-visual signals between participants at different locations to provide visual and voice communication between at least two people in real-time over a network. The term videoconference is meant to be synonymous with terms such as videotelephony, videochat, and more colloquial terminology such as “FaceTiming” or “Skyping.”

The present invention provides an apparatus providing a means for reversible stable association with a portable computing and communications device (PCCD) said apparatus providing an illumination system to illuminate the proximity resulting in illumination of an object in the proximity.

In one embodiment, the invention provides An apparatus to provide illumination of objects for imaging by the camera of a PCCD, said apparatus comprising:

(a) a housing, said housing having a means to provide stable reversible association between the housing and a PCCD,

(b) an illumination system in association with said housing, said illumination system comprising at least one illumination source, said illumination system being arranged to provide illumination of objects in the proximity of said PCCD,

(c) an illumination control system in operable communication with said illumination system, and

(d) a power supply in electrical communication with said illumination system.

The invention further provides an apparatus as described above further comprising a PCCD. The components of the apparatus of the present invention are encased in a three-dimensional housing, said housing having a front side and a reverse side connected by contiguous walls, said housing further providing a means to provide stable and reversible association between the housing and a PCCD such that when said PCCD is in stable association with said housing at least a portion of the front surface of said PCCD is exposed enabling substantially unhindered operation of the camera of said PCCD and enabling substantially unhindered viewing of the display of the PCCD by a person in the proximity.

The housing of the present invention provides a means to stably and reversibly retain a PCCD device while permitting access to the controls of the PCCD necessary for videoconferencing. Stable and reversible association may be achieved by any of a variety of means. Stable reversible association may be achieved or augmented through the use of one or more of recesses or pockets the dimensions of which conform closely to the dimensions of the PCCD. In one embodiment, the housing provides a recess that receives and retains the PCCD while permitting access to the controls of the PCCD necessary for videoconferencing. Stable and reversible association may also be achieved and/or augmented through the use of retaining pins, clips, adhesives (e.g. polyurethane gel), snaps, hook and loop fasteners (e.g. Velcro®), magnets and the like incorporated into the housing. Stable and reversible association between the housing and the PCCD may also be achieved by protrusions from the housing that are received by one or ports provided on the PCCD.

In the embodiment when a recess is provided recess in the front face of the housing to receive a PCCD, the dimensions and shape of the recess in the front face of the housing is designed to closely receive a PCCD so as to restrain the PCCD from transverse movement when associated with the housing yet permit access to the PCCD controls. As used herein, the term “closely receive” is used to describe the difference in dimensions between the inner surface of the recess and the external surface of the PCCD to be received into the recess of the housing or PCCD-specific insert. A PCCD is closely received when there exists a gap between the external surfaces of the PCCD and the internal surface of the recess of less than or equal to 10 mm, less than or equal to 8 mm, less than or equal to 6 mm, less than or equal to 5 mm, less than or equal to 4 mm, less than or equal to 3 mm, less than or equal to 2 mm, less than or equal to 1 mm, or less than or equal to 0.5 mm. In one embodiment, the recess closely receives a PCCD when the inner dimensions of the recess are approximately equal to the outer dimensions of the PCCD to be received with essentially no gap between the PCCD and the inner surface of the recess. When the PCCD is closely received in the recess it is restrained from movement, either in the transverse direction (i.e. movement parallel to the front of the housing) and/or obverse direction (perpendicular to the plane of the front of the housing).

The conformation of the PCCD receiving recess to closely receive the PCCD may be achieved in reference to the external dimension of the PCCD. The external dimensions of PCCD devices are readily determined from examination of the PCCD device or from developer resources provided by the manufacturer. For example, technical specifications for Apple brand PCCDs are provided in a document entitled “Case Design Guidelines for Apple Devices Release R8” Oct. 16, 2014, Apple, Inc. Cupertino Calif. available at: https://developer.apple.com/resources/cases/Case-Design-Guidelines.pdf. Similar resources are available for Samsung PCCD devices at http://developer.samsung.com/resources.

In one embodiment, the housing is constructed of an elastomeric material that facilitates stable reversible association between the PCCD and the housing through deformation of the housing material. In one embodiment where the housing is constructed of elastomeric materials, the housing provides a recess the interior dimensions of which in substantial conformity with the external dimensions of the PCCD enabling the PCCD to be stably and reversibly associated by deformation of the housing material to receive the PCCD.

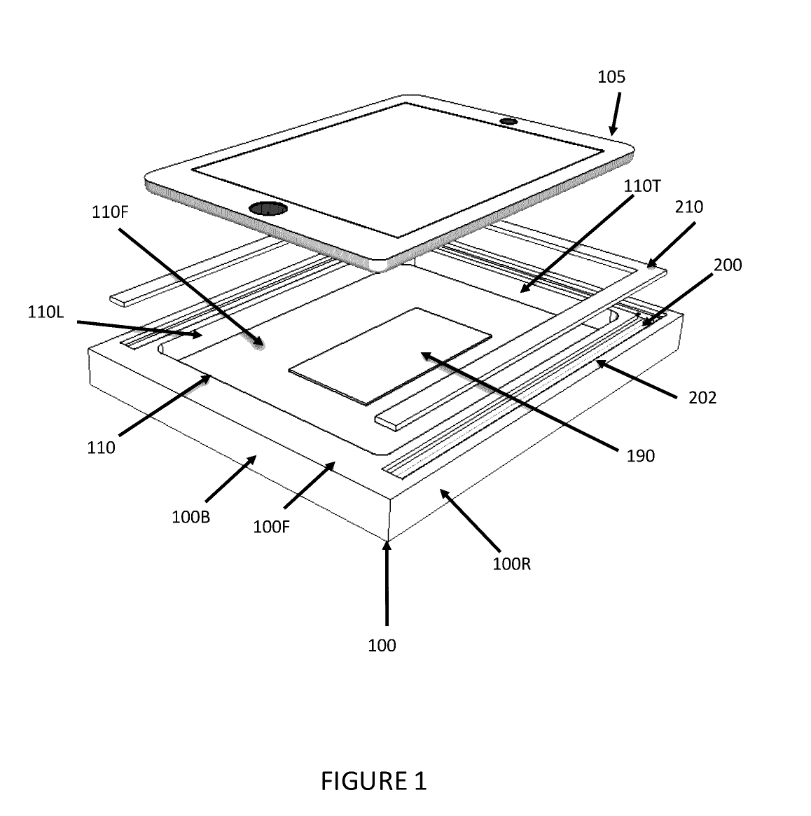

One embodiment of the apparatus of the present invention is illustrated in FIG. 1 of the attached drawings that provides an exploded frontal perspective view to illustrate the various components of the apparatus. FIG. 1 illustrates a housing (100) the exterior of which is defined by a top wall (not shown), a bottom wall (100B), a left wall (not shown), a right wall (100R), a front wall (100F) and an under wall (not shown), a recess (110) to receive a PCCD (105), said recess being defined by a left wall (110L), top wall (110T), right and bottom walls (not shown) and a face wall (110F). The surface of the of front wall (100F) provides a recess (202) to receive the illumination source (200) which is covered by a diffuser (210). As illustrated in this embodiment, the front surface of the recess (110F) is provided with an adhesive pad (190) to provide retention of the PCCD (105) when inserted into the recess (110).

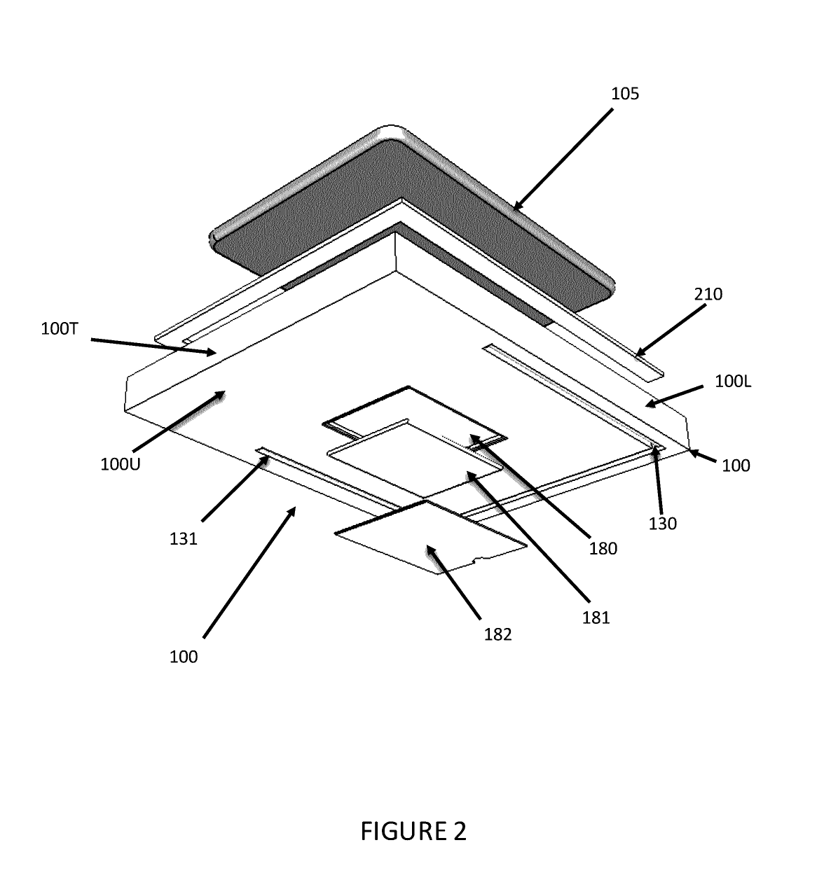

A rear view of the embodiment of the apparatus of the present invention provided in FIG. 1 is illustrated in FIG. 2 of the attached drawings that provides an exploded upper rear view perspective to illustrate the various components of the apparatus. FIG. 2 illustrates a housing (100) the exterior of which is defined by a top wall (100T), left wall (100L), a right wall (not shown) a front wall (not shown) and an under wall (100U) which provides a recess (131) to receive a support (130) illustrated here in retracted position. In this embodiment, the under wall (100U) of the housing (100) is provided with a recess (180) to receive a battery (181) and battery cover (182). Also illustrated here is the optional diffuser (210) that is received by a recess in the front surface of the housing (not illustrated) and a PCCD (105).

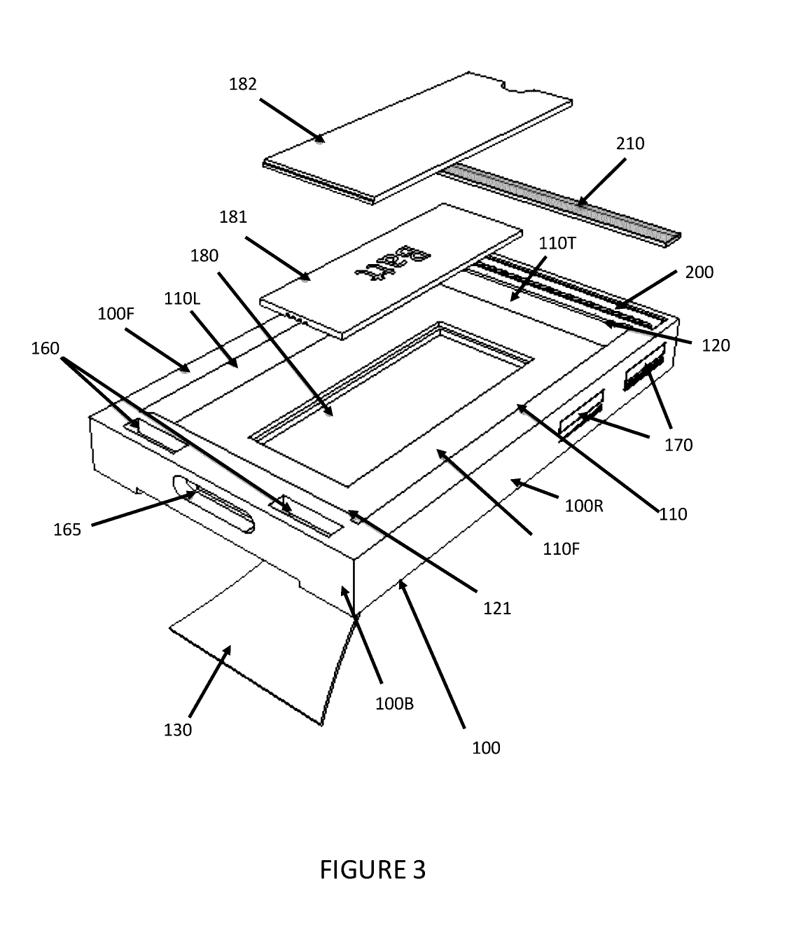

Another embodiment of the invention is illustrated in FIG. 3 of the attached drawings in exploded form viewed from the lower front perspective. Illustrated in FIG. 3 are bottom wall (100B), front wall (100F), and right wall (100R) of the housing (100), the left wall (110L), top wall (110T), and face wall (110F) of the recess (110), the face wall (110F) providing a recess (180) to receive a battery (181) and battery cover (182). The front wall (100F) of the housing (100) provides a recess to receive the illumination source (200) and a diffuser (210). The embodiment of the invention illustrated in FIG. 3 is designed to redirect the sound provided by speakers positioned at the bottom of a PCCD (e.g. iPhone 4S) by providing curved passages (160) which initiate in the lower wall of the recess (not shown) and terminate in the front wall (100F) of the housing (100). One of skill in the art will appreciate that such passages may readily configured to conform to speakers which are provided at any of a variety of locations on the PCCD device. The embodiment of the invention illustrated in FIG. 3 also provides a passage (165) to permit a cable (not shown) to be inserted through the body of the housing (100) from the outer surface of the lower wall (100B) and penetrating the lower wall of the recess (110B) and into a corresponding jack in the bottom of a PCCD (e.g. iPhone 4S, not shown). The figure also illustrates the position of external illumination control switches (170). FIG. 3 also illustrates the position of retention tabs (120 and 121) each extending perpendicular to the top wall (110T) and bottom wall, respectively, of the recess (110). Tabs 120 and 121 assist with stable retention of the PCCD (not shown) when inserted into the recess (110). Also shown in extended position is a support (130).

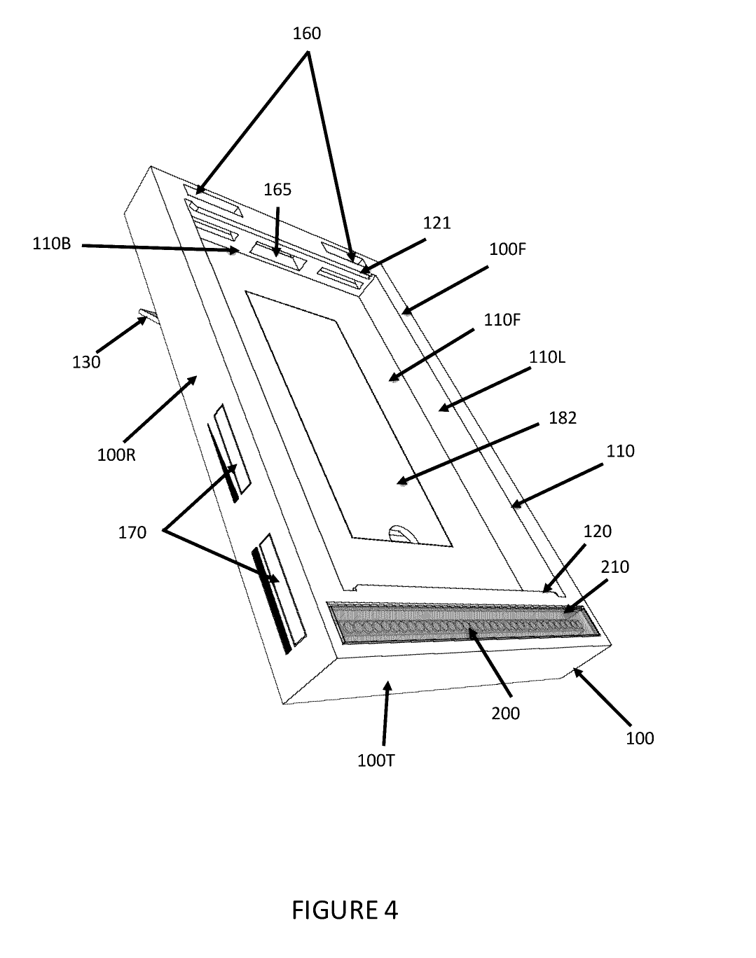

FIG. 4 of the attached drawings provides an upper front perspective view of an embodiment of the invention similar to that provided in FIG. 3. FIG. 4 illustrates the assembled form of the apparatus illustrating the top wall (100T), front wall (100F), and right wall (100R) of the housing (100), the left wall (110L), bottom wall (110B), and face wall (110F) of the recess (110), upper retention tab (120, lower retention tab (121), battery cover (182), illumination source (200) and diffuser (210), sound reflecting passages (160), cable port (165), support (130), and illumination control switches (170).

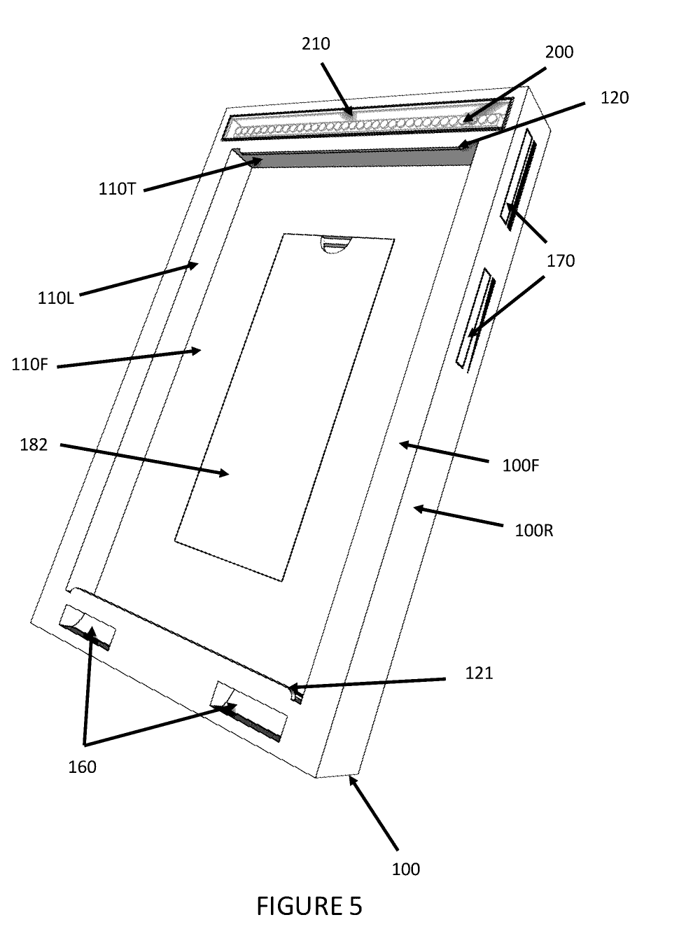

FIG. 5 of the attached drawings provides a right front perspective view of an embodiment of the invention similar to that provided in FIGS. 3 and 4. FIG. 5 illustrates the assembled form of the apparatus illustrating the front wall (100F) and right wall (100R) of the housing (100), the left wall (110L), bottom wall (not shown), and face wall (110F) of the recess (110), upper retention tab (120, lower retention tab (121), battery cover (182), illumination source (200) and diffuser (210), sound reflecting passages (160), and illumination control switches (170).

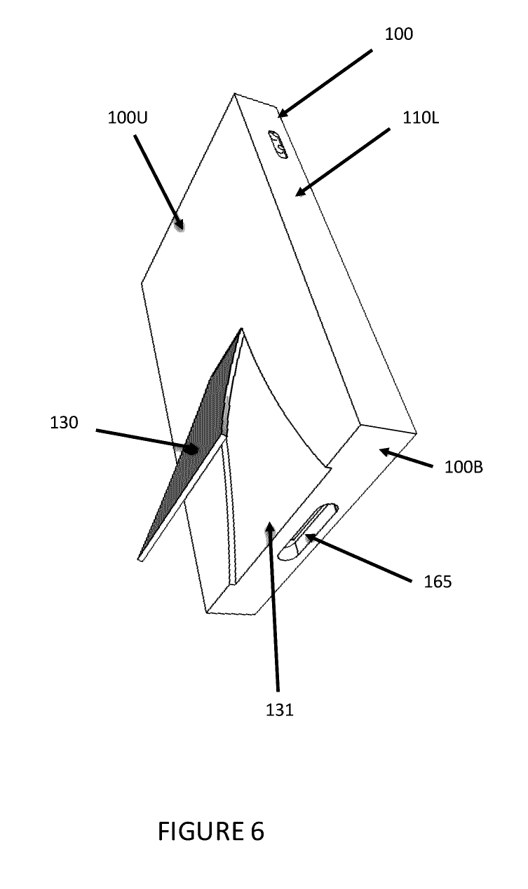

FIG. 6 of the attached drawings provides a lower left rear perspective view of an embodiment of the invention similar to that provided in FIGS. 3, 4 and 5. Illustrated in Figure of 6 is an embodiment of the support (130) which is pivotally attached to the under face (100U) of the housing (100) and is received by the support recess (131) when retracted. Also illustrated in FIG. 6 are the left wall (100L) and bottom wall (100B) of the housing (100) and cable port (165).

In another embodiment of the invention, rather than the housing providing a recess configured to receive only a particular PCCD, the housing may be designed to receive a “PCCD-specific insert” that may be reversibly associated with the surface of the housing, such PCCD specific insert providing a recess that stably and reversibly receives a PCCD. A PCCD-specific insert refers to the embodiment where the housing is designed in one configuration capable of receiving a PCCD insert of standard dimensions wherein the insert is designed to stably and reversibly retain a PCCD of a particular conformation. In this manner, a single housing can be used with a variety PCCD devices through the use of different PCCD-specific inserts. In such manner, a single housing would be useful in conjunction with a variety of PCCDs, each PCCD being associated with a PCCD-specific insert. For example, a single housing design would be able to receive a PCCD-specific insert that is designed to stably and reversibly retain an iPhone 5 model PCCD as well as a PCCD-specific insert that is designed to stably and reversibly retain an iPad Mini PCCD.

The PCCD-specific insert is also designed for stable and reversible association with the housing. Stable and reversible retention of the PCCD-specific insert may be achieved through any of a variety of means understood to those of skill in the art such as adhesives, snaps, hook and loop fasteners, conventional fasteners (e.g. screws), locating pins and corresponding recesses or apertures in the PCCD or surface (or vice versa), magnets positioned appropriately in the surface of the housing and/or PCCD-specific insert (or vice versa), and the like.

In one embodiment, the PCCD-specific insert provides a recess that closely receives a PCCD resulting in stable and reversible association between the PCCD and the PCCD-specific insert.

In one embodiment, the PCCD-specific insert is constructed of an elastomeric material that facilitates stable reversible association between the PCCD and the PCCD-specific insert through deformation of the PCCD-specific insert material. In one embodiment where the PCCD-specific insert is constructed of elastomeric materials, the PCCD-specific insert provides a recess the to closely receive the PCCD enabling the PCCD to be stably and reversibly associated by deformation of the PCCD-specific insert material to receive the PCCD.

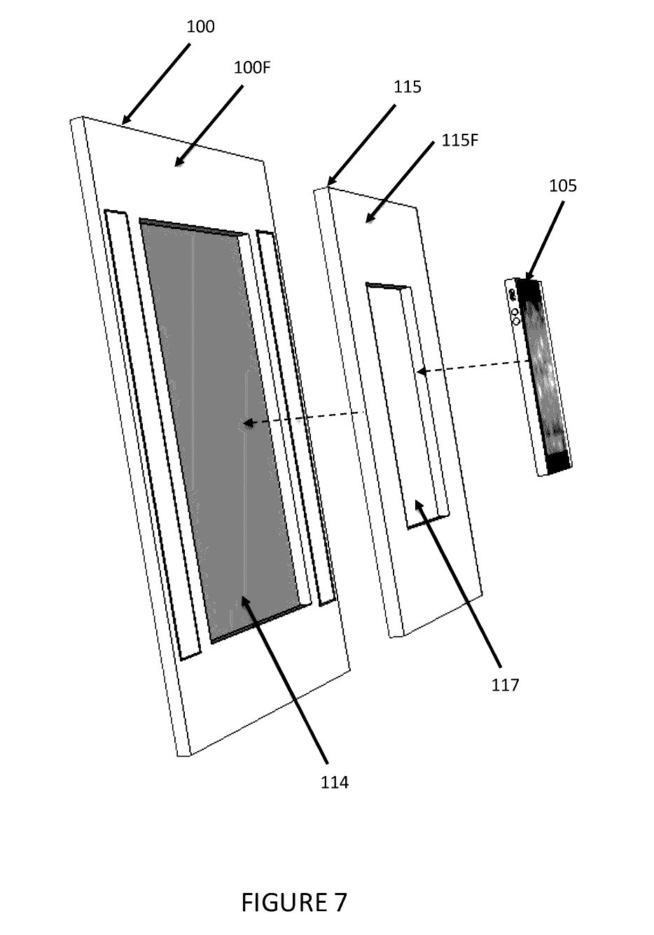

An illustrative embodiment of an exploded configuration of the apparatus of the present invention is provided in FIG. 7 of the attached drawings wherein the front wall (100F) of the housing (100) is provides a recess (114) to reversibly receive a PCCD-specific insert (115), the front wall (115F) of said PCCD-specific insert (115) providing a recess (117) that conforms substantially to the external dimensions of the PCCD (105) such that when the insert (115) is associated with the housing (100) the PCCD (105) is maintained in stable association with the housing, the dashed arrows indicating the direction of insertion of the PCCD (105), PCCD specific insert (115) relative to the housing (100).

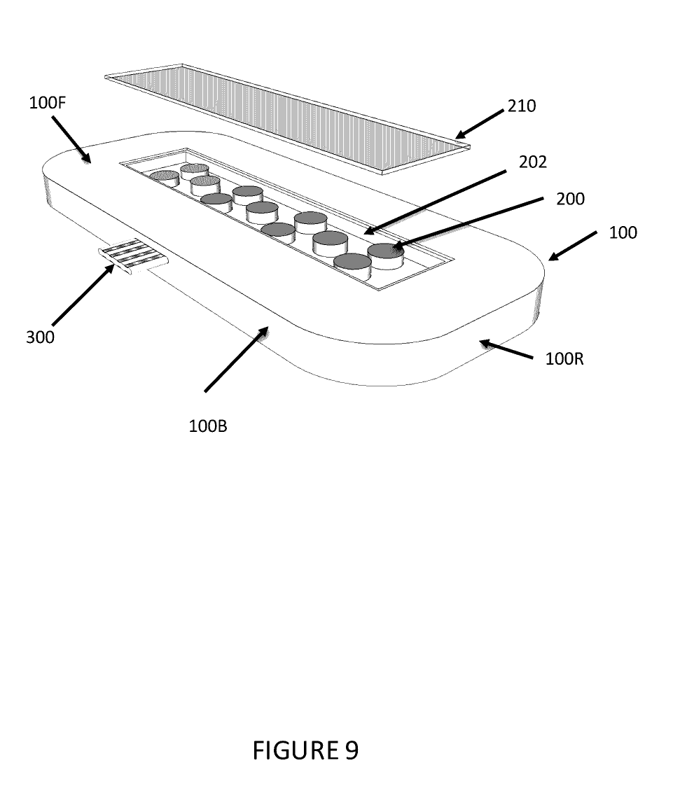

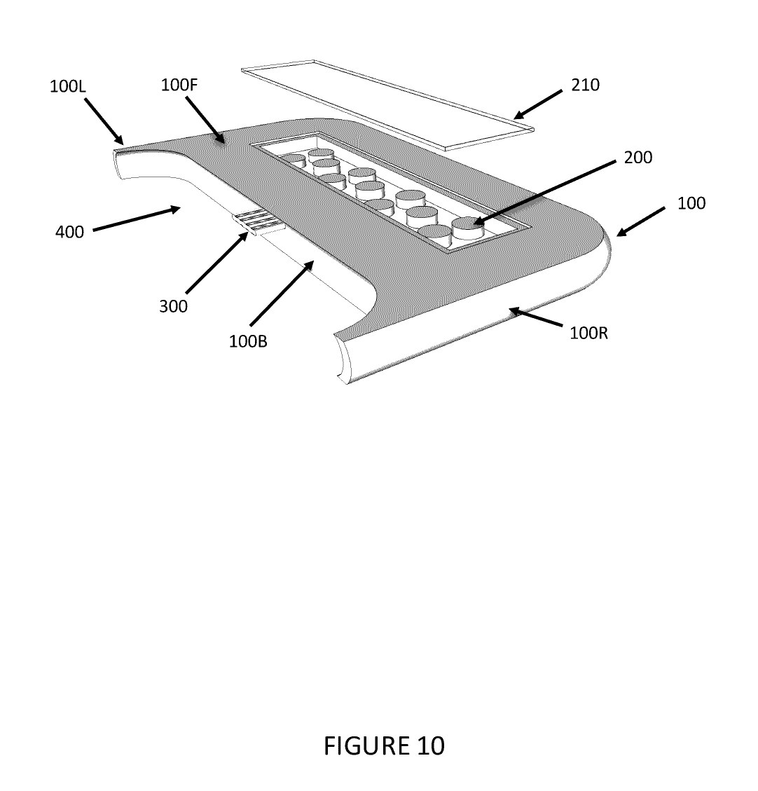

Alternative embodiments of the invention are illustrated in FIGS. 9-10. FIG. 9 provides a perspective drawing of one embodiment of the invention illustrating the housing (100) having a front wall (100F), a under wall (not shown), top wall (not shown), right wall (100R), bottom wall (100B), a recess (202) containing the illumination source (200), diffuser (210) and a male plug (300) extending from the bottom wall (100B) of the housing (100) which is to be received into a corresponding female port of the PCCD (not shown) providing stable association between the housing (100) and the PCCD (not shown). In one embodiment, the male plug is simply a structural feature that is received into a female port provided on the PCCD so as to provide stable and reversible association between the apparatus and the PCCD. Alternatively, as previously described, the male plug may optionally provide electrical communication between the PCCD and the other components of the apparatus, in particular the illumination system. Where the male plug provides electrical communication between the apparatus and the PCCD, the apparatus may optionally draw power from the power supply of the PCCD power the electrical components associated with the housing including but not limited to the speaker(s), microphone(s) illumination source(s), amplifier(s), and/or communications module(s) and avoiding the need to provide an additional power supply. Alternatively, or in addition, the male plug may be used to associate with a charging system to recharge a power supply contained within the housing. A similar embodiment is provided in FIG. 10 of the attached drawings illustrating a housing (100) having a bottom wall (100B) and extended left wall (100L) and extended right wall (100R) to define a recess (400) to receive a portion of the PCCD device. As illustrated the bottom wall (100B) provides a negative curvature to receive a portion of the device. A housing so configured is designed to receive at least a portion of the device, the recess providing enhanced stability of association between the housing and the PCCD.

The apparatus of the present invention comprises an illumination system, said illumination system being arranged to provide illumination of objects in the proximity of the apparatus, said illumination system comprising at least one illumination source in electrical communication with a power supply and a control system to provide for controlling the output of the illumination source. The apparatus is configured such the illumination source(s) provides illumination of the region proximal to said apparatus. This may be achieved by placing the illumination source at or near the front surface of the housing so as to provide illumination of the proximity of the apparatus. Alternatively, the light source may be incorporated in the housing of the cavity and the light directed to the area in front of the apparatus using reflective surfaces incorporated into the body of the housing to redirect the light to the proximity of the front of the apparatus.

The term “illumination source” as used herein refers a device that converts electricity into electromagnetic radiation. Examples of illumination sources suitable for use in the practice of the present invention which provide illumination in the visible spectrum include but are not limited to one or more incandescent, fluorescent or LED lamps including combinations of one or more of said illumination sources. Illumination sources useful in the practice of the present invention are commercially available from a wide variety of suppliers and conformational adaption of the housing to accommodate such various illumination sources is readily apparent to the skilled artisan. The illumination source is in electrical communication with the power supply through one or more conductors.

In one embodiment, the illumination source is comprised of one or more light emitting diodes (LEDs). As compared to conventional incandescent or fluorescent illumination sources, LED illumination sources provide an advantage of low power consumption that may be advantageous in situations where the power supply for the apparatus is a portable power supply such as a battery. LEDs also provide light of high intensity and have a long service life. In one embodiment where LEDs are employed, the LEDs are dimmable LEDs providing for variable light output. The apparatus of the present invention may provide various illumination sources providing illumination of various colors and/or wavelengths to enable modulation of the properties of the illumination.

LED illumination sources are available in a variety of color temperatures, output, operating voltages and physical configurations including elongated tube lights and strip lights, particularly strip lights having red, green and blue LEDs (RGB LED strip lights). Variable output LED strip lighting is commercially available from a variety of suppliers including OSRAIVI Sylvania, Philips, and General Electric. One example of a commercially available LED tube light is the Seesmart Model No. 200621 tube light comprising 30 LEDs providing an output of approximately 204 lumens at a color temperature of 4000K-4500K (commercially available from Seesmart, Inc., 4139 Guardian Street, Simi Valley Calif. 93063). Fixed and variable color LED lighting systems are available from Philips Color Kinetics (3 Burlington Woods Drive, Burlington MA 01801) marketed under the Color Kinetics®, including but not limited to the ColorGraze, Essential Color (eColor), Vaya Cove Color, Vaya Linear, Vaya Linear Color, Vaya Linear MP, Vaya Linear MP RGB.

As used herein, the term “illumination” refers to electromagnetic radiation produced by an illumination source. Illumination is described with respect to a variety of factors including but not limited to color temperature, intensity, and frequency.

In the typical practice of the present invention, the illumination provided by the illumination source illuminates the object to be viewed by the camera of the PCCD with a light that is as natural as possible. Illumination sources having a higher CRI Ra value are preferred, the CRI Ra value of 100 being that illumination provided by standardized daylight. Fluorescent illumination sources typically produce illumination having a CRI Ra value from about 50 to about 90. LED illumination sources can conventionally produce illumination having a CRI Ra of greater than 80. In one embodiment of the invention, the illumination source has a CRI Ra value of from 50 to 100, 60 to 100, 70 to 100 or 80 to 100, 90 to 100 or 95-100.

A factor that affects an individual's appearance when being imaged by camera of a PCCD is that the ambient lighting, particularly fluorescent lighting, is perceived as providing a non-realistic image of the individual due generally to what is an excessive color temperature. So-called “warmer” colors (e.g. reds, oranges) are actually lower on the color temperature scale as opposed to “colder” colors (e.g. blues, greens). Color temperature is measured on the Kelvin scale. Conventionally, human beings perceive “natural” coloration under incandescent light where having a color temperature ranging from approximately 2400K to 7500K. It is generally accepted that most individuals find a white balance in the range of 2000K to 6500K most realistic. Further, the color temperature of the illumination emitted by the illumination source may be fixed or variable. Variability in color temperature may be achieved by controlling the degree of illumination of and/or the number of each of said illumination sources or through the use of filters.

In one embodiment, variability in color temperature may be accomplished by a combination of illumination sources each of which provides a particular color temperature light. Variable illumination may be achieved by differentially energizing the various illumination sources via the illumination control system to provide illumination of an object in the proximity of the apparatus of particular color temperature. Alternatively, color temperature may be varied by differentially energizing illumination sources of different colors. A particular color temperature may be achieved by providing variant energy to the illumination source comprising illumination sources of two or more different colors (typically at least blue and red) and using the illumination control system to independently vary the energy provided to each colored illumination source so as to enable a blending of colors to achieve a desired color temperature.

In another embodiment, variability in color temperature may be achieved by the use of one or more filters positioned between the illumination source and the subject to be imaged by the camera. As used herein, the term filters include diffusers or clear or translucent colored elements (e.g. clear or translucent colored films) that alter the color temperature of the illumination that is generated by the illumination source prior to being projected to the user. Filters may be employed to adjust the color temperature, frequency and/or intensity of the illumination provided.

In one embodiment of the invention, the illumination source(s) of the apparatus provide illumination of an object in the proximity of the apparatus with light in the visible spectrum having a color temperature of from about 2000 to about 6500K, alternatively from about 2400 to about 5500K. In one embodiment of the invention, the illumination source provides illumination in the visible spectrum having a color temperature between 2000K and 4500K, alternatively between 2400 and 4000K, or alternatively between 2500K and 3600K.

The illumination source of the present invention provides illumination of an object in the proximity of the apparatus of an intensity appropriate for imaging by the camera. The intensity of illumination of a subject provided by a given illumination source is a function both of the output of the illumination source and the distance from the illumination source. In one embodiment, the illumination system of the present invention provides light in the visible spectrum having an intensity of illumination of an object located in the proximity the apparatus of at least 30 lux, optionally at least 50 lux, optionally at least 100 lux, optionally at least 200 lux, optionally at least 300 lux, or optionally at least 400 lux. The determination of lux may be readily determined by a variety of lux meters commercially available from a wide variety of sources such as the Model LX1330B Digital Illuminance/Light Meter (commercially available from Amazon.com as item number BOO5A0ETXY).

The electromagnetic energy produced by the illumination source may provide electromagnetic radiation in the visible spectrum including but not limited to visible light of one or more frequencies, colors, white light, or electromagnetic radiation not of the visible spectrum including but not limited to ultraviolet light or infrared light. The illumination source may provide multiple sources of illumination including illumination in the visible spectrum, the infrared spectrum and/or the ultraviolet spectrum. In one embodiment, the illumination source of the apparatus provides multiple illumination sources (e.g. LEDs) each of which emit electromagnetic radiation in the visible spectrum, the infrared spectrum, and/or the ultraviolet spectrum. LEDs that emit light in the visible spectrum, the infrared spectrum, and the ultraviolet spectrum are commercially available from a wide variety of suppliers. In particular the illumination source used in the practice of the present invention may provide illumination of one or more of ultraviolet-A, ultraviolet-B, ultraviolet-C, near infrared, far infrared, long wavelength infrared, mid-infrared frequencies.

The apparatus of the present invention further provides an illumination control system in operable communication with said illumination system. By the term “operable communication” is meant that the control of the illumination source may be either through conventional electrical conductors (electrical communication) or through wireless communication with the PCCD. The illumination control system may provide a fixed or variable output from the illumination source.

In a simple form, the illumination control system is a single throw (on/off) switch in electrical communication with the power supply and the illumination source. In an alternative embodiment of the invention, the illumination control system provides a variable level of illumination. Such variable level of illumination may be provided through the use of conventional variable electronic switches and circuits switched or variable by external means (e.g. a “dimmer” switch) to achieve a particular level of illumination. The control of the lighting system color and intensity may be provided in conjunction with the housing may also be provided on a wired or wireless remote control. As previously described when illumination sources of multiple types are provided, the variable illumination control system may be configured to provide enhanced output from an illumination source of one color relative to another. Examples of variable switch systems that may be used in the practice of the present invention include the Vaya Touch DMX512 controller (commercially available from Philips Lighting, 3 Burlington Woods Drive, Burlington Mass. 01801).

In another embodiment of the invention the illumination control system is a control module in in operable communication with the PCCD and electrical communication with the illumination source and the power supply. Conventional PCCDs are equipped with wireless communication systems. Wireless communication systems transmit and a receive data with compatible devices in accordance with wireless communication protocols such as Wi-FiTM 802.11 a/b/g/n, Bluetooth®, Near Field Communication (NFC) or cellular data transfer protocols such as GSM®, 3G, 4G, LTE and similar standard protocols for the wireless transfer of data. These PCCD functions may be employed for the communication of information received through the image sensor of the camera of the PCCD to the control module. In an alternative embodiment, the PCCD may communicate with the control module via a conventional wired connection such as through a male plug that is received into a port (e.g. USB, microUSB, USB-C, Lightning port, 30 pin connector) of the PCCD.

A control module may comprise a wireless communication module (e.g. WiFi, near-field communication (NFC) or Bluetooth module) and a microprocessor in electrical communication with the wireless communication module, said microprocessor programmed to perform one or more computational functions on the information received from the PCCD via the wireless communication module and to provide an output signal to the illumination source, said output signal producing a variation in the intensity, frequency and/or color temperature of the illumination provided by the illumination source. For example, PCCD cameras incorporate light meters that adjust exposure to the perceived lighting conditions. The control module microprocessor is programmed to interpret such light meter data received wirelessly from the PCCD and provide an output signal to the illumination source so as provide illumination of an object in the proximity of the apparatus by the illumination source.

The control module may be the microprocessor of the PCCD device when running software that is designed to communicate with the apparatus. In one embodiment, a software program resident on the PCCD device is provided which is in operable communication with the apparatus (e.g. by physical connection) enabling the operator to control the illumination and other functions of the apparatus using the software resident on the PCCD device to control the illumination system and other functions through such software. In such a configuration, the microprocessor of the PCCD may be the illumination control system.

The control module may also be remotely operated by software in communication with the apparatus of the present invention. For example, as discussed in more detail below, an operator at a remote location may use networked software to communicate with the PCCD of the end user which in turn activates one or more functions of apparatus that may provide therapeutic or diagnostic applications.

The illumination source(s) of the illumination system is/are in electrical communication with a power supply via the illumination control system. The power supply for the illumination source may be provided through conventional conductive materials in electrical communication with an external power supply such as conventional wall sockets through the use of an electrical cord. When conventional wall current is used as a power source, the apparatus may provide a transformer or power adapter to convert wall current into appropriate current for the illumination source employed.

In order to facilitate portability, the power supply may be incorporated into the housing using a portable power supply (e.g., one or more batteries or solar cells). In one embodiment of the invention, the apparatus comprises an integrated, self-contained power supply. Such self-contained power supply may comprise one or more batteries. The battery(ies) may be non-rechargeable or rechargeable. Examples of batteries useful in the practice of the present invention include zinc-carbon, zinc-chloride, alkaline (e.g., zinc-manganese dioxide), lithium (e.g., lithium-copper oxide, lithium-iron disulfide, lithium manganese dioxide), silver oxide, silver-zinc, NiCd, NiMH, NiZn, lithium ion batteries. The term battery includes a single cell or a plurality of two or more cells. The apparatus may also incorporate or be provided with a solar collector for charging the battery source. When one or more rechargeable batteries are employed as the power source, the device of the present invention may also be provided with a external charging system appropriate for the particular configuration and specifications of the rechargeable battery employed and the housing may also provide a plug system for reversible association of the external charging system with the housing containing the rechargeable batteries.

In another embodiment of the invention the power supply used to power the electrical components of the apparatus is the power supply of the PCCD device. In this configuration, the apparatus provides a means of electrical communication with the PCCD device to draw power from the power supply of the PCCD device. Typical examples would include the use of an electrical connector adapted for the particular PCCD device (e.g. micro-USB) that enables the draw of power from the device. In PCCD devices incorporating wireless charging systems, the apparatus may use such charging system in reverse to draw power wirelessly from the PCCD power supply.

The apparatus of the present invention may be constructed of any of a variety of materials. Examples of materials suitable for use in construction of the housing and supports include metals, plastics, wood, compressed paper, glass, rigid foams, cardboard, glass and/or ceramics. The housing, PCCD-specific insert (if employed) and/or supports may be constructed from the same materials or may be produced from different materials.

Examples of metals useful in the construction of the apparatus of the present invention include but are not limited to steel and steel alloys (e.g. stainless steel), aluminum and aluminum alloys, and magnesium and magnesium alloys. Examples of plastic materials useful in the construction of the housing and/or supports are well known in the art and the techniques involved in the forming of the materials into the housing are apparent to the skilled artisan. The term plastics includes but is not limited to polycarbonate (PC), polyethylene (PE), high density polyethylene (HDPE), polyetherimide (PEI), polysulfone (PSO), polyethersulfone (PES), polyethylene terepthalate (PET), polypropylene, polystyrene, high impact polystyrene (HIPS), acrylanitrile butadiene styrene (ABS), polyvinylchloride (PVC), acetal, Nylons (e.g., Nylon 4-6, Nylone 6-6, Nylon 11, or Nylon 12), acrylic-styrene-acetonitrile (ASA), polyester liquid crystal polymer (LCP), stylene acrylonitrile (SAN), polyvinyldiene difluoride (PVDF), melamine, phenolics and the like. The housing, PCCD-specific insert and any supports may also be constructed of composite materials such as sandwich-structured composites.

In general, when selecting materials for construction of the apparatus of the present invention, it is generally preferred to employ materials that do not significantly impair the ability of communication functions (e.g. cellular, Bluetooth®, Near-field Communications (NFC) or WiFi® systems) of the PCCD. Examples of such materials that minimize interference with the communication functions of the PCCD include but are not limited to non-ferrous metals, plastics, paper, wood, and ceramics and composites thereof. The following materials should be avoided or minimized in the construction of the housing: metals (e.g. steel, aluminum, magnesium, titanium, etc.), plastics with significant carbon content, plastics with significant glass content, plastics with metallic plating, metallic paints or coatings, black paints with high carbon loading, white paints which high titanium dioxide loading and metallic physical vapor deposition coatings.

Typical procedures for forming the materials are well known to those of skill in the art and include conventional processes such as compression molding, blow molding, casting, extrusion, pressure forming, and the like. The techniques for forming such plastic materials into the conformations of the present invention are well known to those of skill in the art (see e.g. Olmsted and Davis (2001) Practical Injection Molding, Marcel Dekker New York; Rosato and Rosato (1986) Injection Molding Handbook; Van Nostrand Reinhold Company, New York). The elements of the apparatus including the housing, PCCD-specific insert (if employed) and/or supports may also be produced by deposition forming (often referred to as “3D printing”) using conventional materials such as plastic, metallic or metallic precursors. Metals may be formed using conventional techniques such as stamping, casting, machining, forging (including powder forging), hydroforming, thermoforming, compression molding, or the like. Such composite materials may be formed using conventional contact molding technologies such as hand lay-up or spray lay-up technologies.

The housing, PCCD-specific insert (if employed) may further incorporate structural features to improve the rigidity of the housing and/or facilitate the attachment of other components. Such structural features enable the use of less material and facilitate portability by minimizing the weight of the apparatus. Examples of such structural features include ribs or bosses. Such structural features may be incorporated into the initial manufacture of the housing components or may be incorporated in additional manufacturing steps.

In some instances, it may be desirable to provide a finish to the materials to minimize corrosion from atmospheric or operator sources or to improve durability, handing characteristics or appearance. Optionally, the materials may be electroplated, painted, dip-coated, or flocked to enhance appearance and/or durability. Optionally, portions apparatus in contact a support surface may also coated or fitted with non-slip materials.

As illustrated in FIG. 1 of the attached drawings, the apparatus of the present invention may incorporate one or more of supports to maintain the apparatus at an acute angle relative to a supporting surface, said supports being operably associated with the housing to maintain the housing at a fixed angle to the surface on which the apparatus is placed.

The support(s) may be conventional supports which are retractable by means of being rotatably, pivotally or hingedly attached to the housing to enable them to be reversibly moved the user from a first retracted position to a second extended position thereby providing the PCCD device at an angle position relative to a support surface. The supports communicate with the structure through the use of conventional linkages enabling rotatable, pivotal, or hinged linkages through the use of swivels, pivots, pins, and/or hinges (including active hinged mechanisms). Supports may be of any general cross-section in design and may be solid, hollow, or open such as through the employment of tubular, three sided U-shaped channel, or two-sided angled materials.

In general, when the supports are pivotally attached to the housing, when extended they are generally provided at an included angle of about 90 degree or less. The support/housing interface may employ the use of one or more detents to retain the support from further movement at a given angle relative to the housing to receive and position the supports at an angle relative to the underside of the housing. The housing may also provide means to retain the supports in a contracted position through the use of conventional clamps, spring clips, magnetic contacts, friction, detents, pins, and the like.

In one embodiment of the invention, one or more supports may be configured as a substantially planar panel hingedly associated to the reverse side of the housing.

In another embodiment, the support is provided by a single substantially planar panel with a triangularly shaped foot enabling placement of the apparatus in the portrait or landscape configuration. Alternatively, the supports may be provided separate from the housing, the housing being designed to reversibly and stably receive the supports, such stable and reversible retention achieved through the use of threaded or close fitting cavities integrated into or associated with the housing.

The integrated speaker systems of many PCCD devices provide marginal audio output making it difficult in some situations to rely on the integrated speaker systems. This is particularly an issue for users having impaired hearing such as the elderly. Studies have shown that individuals are less likely to participate in videoconferencing if they are required to wear a headset. Consequently, in addition to the provision of illumination, the apparatus of the present invention may optionally provide enhanced audio through the use of one or more auxiliary speakers associated (either incorporated into the housing or remotely located and in operable communication with the apparatus) and in operable communication with the PCCD. To provide a more natural audio experience, it is desired that the speakers be associated with the housing (preferably on the front face of the housing) near the PCCD display. The apparatus may also provide a means to amplify the output signal (e.g., and amplifier) communicated to the speaker(s) to provide for enhanced volume levels, optionally providing a variable switch to control the output level of the speaker(s).

As previously discussed in relation to the illumination control system the term operable communication contemplates either conventional wired or wireless communication between the PCCD and the speakers. Such wired communication may be achieved by associating the speakers with the conventional 3.5 mm headphone jack of the PCCD or via an integrated port (e.g, micro-USB, Apple “30 Pin”, Apple Lightning).

When wireless communication is desired, operable association between the PCCD and the speaker system is mediated by a control module comprising a wireless communication module (e.g. a Bluetooth module) and a microprocessor programmed to interpret information received from the wireless communication module to provide an output signal to the speaker system. Where the illumination system (or one or more other features of the apparatus) is mediated by a control module in wireless communication with the PCCD, a combined control module may also be programmed to process audio signals for output to the speakers in addition to the other functions.

The housing may also incorporate structural features to redirect and/or amplify the sound produced by the PCCD device's speaker systems, such as to redirect the speakers at the bottom of the iPhone 4S to project forward toward the observer.

The integrated microphones of many PCCD devices are typically marginal for use at distances common in a videoconferencing. The alternative is to use a headset with an auxiliary microphone positioned close to the mouth of the user. However, as previously discussed, studies have shown that individuals are less likely to participate in videoconferencing if they are required to wear a headset. Consequently, the apparatus of the present invention may optionally provide enhanced voice reception through the use of one or more auxiliary microphones associated with the housing and in operable communication with the PCCD. To provide a more user experience, it is desired that the microphone(s) be placed in the housing (preferably on the front face of the housing) near the PCCD display. The microphone is typically directionally positioned so as to optimize the receipt of sound from the user. Directional microphones may be employed. The apparatus may also provide a means to amplify the output signal provided by the microphone to the PCCD, optionally providing a variable switch to control the input level of the microphone(s) to the PCCD. The microphone may be a directional high-sensitivity microphone, optionally incorporating active or passive noise cancelling functions, enabling accurate audio capture as well as shielding from extraneous noise.

As previously discussed in relation to the illumination control system the term operable communication contemplates either conventional wired or wireless communication between the PCCD and the microphone. Such wired communication may be achieved by associating the speakers with the conventional 3.5 mm headphone jack of the PCCD or via an integrated port of the PCCD.

When wireless communication is desired, operable association between the PCCD and the microphone(s) is mediated by a control module comprising a wireless communication module (e.g. a Bluetooth module) and a microprocessor programmed to interpret information received receive the output signal from the microphone and transmit the information to the PCCD device for communication to remote videoconference participants. one or more other features of the apparatus are mediated by a control module in wireless communication with the PCCD, a combined control module may also be programmed to process audio signals from the microphone(s) in addition to the other functions.

In one embodiment, the apparatus may provide a light meter in operable communication with the illumination control module. In one embodiment, the light meter is associated with the housing, the sensing means of the light meter is positioned in association with an aperture in the front side of the housing and the axis of said light meter sensing means being substantially coaxial with the line of sight of the camera when a PCCD device is associated with the housing.

The apparatus of the present invention may optionally provide for a means to provide electrical power to the PCCD when in association with the housing. Such function may be accomplished by electrical communication between the power supply of the apparatus and the PCCD mediated by male connector (e.g micro-USB, Apple 30-pin or Lightning plug) provided in association with the housing in an orientation appropriate to the configuration of the particular PCCD. Alternatively, the apparatus may optionally provide a wireless charging system in association with the housing to provide wireless charging to those PCCD devices that accommodate wireless charging. The charging system may incorporate a wireless charging pad operating in accordance with standard wireless charging protocols such as the Qi standard.

The front facing camera of a PCCD is typically incorporates a wide-angle lens. Wide-angle lenses can result in distorted images resulting in a non-natural appearance of the subject. For example, using a camera with a wide-angle lens to photograph a person relatively close to the lens, the nose, eyes, lips and face appear unrealistically large. The apparatus of the present invention may optionally include one or more lenses to correct optical distortions such as barrel distortion. Correction of wide-angle lens distortion may be accomplished by the apparatus of the present invention positioning an image correcting lens between the object to be imaged by the camera and the camera. In one embodiment, said image correcting lens is incorporated into the housing such that when the PCCD is stably associated with the housing, the lens is positioned over the front facing camera aperture providing lens. Alternatively, the lens may be provided on an armature enabling positioning of the lens over the front facing camera aperture providing lens to as to provide correction of the wide angle lens distortion. Correction of wide-angle lens distortion may also be accomplished by software, resident on or in communication with the PCCD.

Image sensors provided on typical PCCD devices are capable of detecting light outside the visible spectrum (wavelengths from approximately 400 nm to 700 nm). To facilitate enhanced detection of light in the invisible spectrum, the apparatus of the present invention may optionally provide a filter element to be disposed over the camera of the PCCD to selectively allow/restrict the transmission of light of a particular frequency range. For example, the apparatus may provide an infrared filter that restricts the majority of transmitted light to be restricted to the infrared spectrum. Alternatively, the apparatus may provide an ultraviolet filter that restricts the majority of transmitted light to be restricted to the ultraviolet spectrum.

The invention further provides a software capable of execution on a PCCD or an external computing device in operable communication with the PCCD, said software application directing the control of the illumination system of the apparatus of the present invention, and optionally the audio output and/or input controls. The software application may exist in one or more of series of modules, each module be capable of separable implementation. The application software is accessed from within or loaded into the PCCD device or external computing device in communication with the PCCD. Computer-readable media in which such formatted data and/or instructions may be embodied include, but are not limited to, a tangible memory (e.g., random access memory, read only memory, memory cards, hard drives, etc.) to be read by a central processing unit to implement the functions.

As disclosed herein, embodiments and features of the invention may be implemented through computer-hardware, software and/or firmware. Although some of the disclosed implementations describe components such as software, systems and methods consistent with the innovations herein may be implemented with any combination of hardware, software and/or firmware.

In one embodiment, the initiation of the software may place the PCCD device (and/or remote devices in communication with the PCCD) in condition for display of the image to be captured by the image capture system of the PCCD on the PCCD display or the display of the remote device. For example such software may place the PCCD device in photo or video mode, energize the illumination system, and enable controls such as illumination level, zoom control, exposure, and/or focus control of the lens of the PCCD device, recording, and broadcast of the signal to remote devices such as other networked computers and portable computing and communication devices. Additionally, software subroutines to suppress conflicting signals (such as incoming telephone calls, text messages, e-mails and the like) can be incorporated into the code to preclude interruptions of the display of image sensor signal and the view to the operator.

As noted in several reports, a primary obstacle to the adoption of videoconferencing, particularly in the home, is a concern for privacy due to the fact that the remote videoconference participant is frequently capable of visualizing not only the user participating in the videoconference but objects and people in the background. Software has been developed and is commercially available that provides for background image blurring to avoid the visualization of the environment other than the videoconference participant. The software of the present invention may incorporate such background image blurring code.

The present invention further provides a method of providing enhanced illumination to an individual participating in a PCCD mediated videoconference said method comprising the steps of:

a) providing an apparatus to provide illumination of objects for imaging by the camera of a PCCD, said apparatus comprising:
i. a housing, said housing having a means to provide stable reversible association between the housing and a PCCD,
ii. an illumination system in association with said housing, said illumination system comprising at least one illumination source, said illumination system being arranged to provide illumination of objects in the proximity of said PCCD,
iii. an illumination control system in operable communication with said illumination system, and
iv. a power supply in electrical communication with said illumination system.

b) inserting a PCCD device into said apparatus, said PCCD device having videoconferencing software installed;

c) running the software on a PCCD device such that the camera of said PCCD is activated.

The invention further provides a method of alleviating social anxiety disorder associated with a participation in a video conference, said method comprising the use of an apparatus as described herein to provide illumination of a subject during a PCCD mediated videoconference. As previously discussed, the adoption of videoconferencing has been hindered by the anxiety produced by individuals who are who feel self-conscious of their appearance on camera. This can be particularly acute in individuals suffering from social anxiety disorder (Diagnostic and Statistical Manual of Mental Disorders (DSM-5), Social Anxiety Disorder, American Psychiatric Association 2013). Such individuals exhibit intensive nervousness and self-consciousness during social situations or even the anticipation of social functions for days or weeks in advance. In some instances, the anxiety rises to a level such that it interferes with an individual's daily routine including school, work and relationships.

Talking on the telephone, even in the absence of video transmission is a common situation that provokes anxiety in these individuals. Videoconferencing heightens this anxiety. However, videoconferencing is being more widespread in use in personal interactions and in the workplace. As a condition of employment it is required that a party participate in such video calls. More and more job interviews are being conducted by home-based (often PCCD mediated) video conferences. A job interview is a particularly stressful situation which is rendered even more stressful by the perception that one's appearance is suboptimal. The apparatus of the present invention mitigates anxiety in individuals who participate in such videoconferences and can facilitate their work and personal interactions.

Additionally, in accordance with the increasing adoption of telemedicine, mental health professionals have employed videoconferencing to interact with patients and provide cognitive behavioral therapy (including those with anxiety related disorders), particularly to patients in remote locations or who are unable to come to the doctor's office. The apparatus of the present invention mitigates anxiety in afflicted individuals who participate in such telemedicine applications and can assist in their therapeutic outcome.

The apparatus of the present invention may be used for remote medical practice (“telemedicine”) including store-and-forward, remote monitoring, real time interaction, remote diagnosis and remote treatment. Such telemedicine applications of the apparatus of the present invention are useful in a variety of medical fields including but not limited to telerehabilitation, telepsychiatry, telepsychology, and teledermatology. One application of videoconferencing is used in the medical community to enable caregivers to interact with patients in remote locations or where the patient is homebound and has difficulty travelling to the medical care facility. In one embodiment of the invention, the apparatus may be employed to transmit images of body parts (such as a hand of human subject). The image data transmitted may be further be analyzed by image analysis software to identify potential disease states. The present invention further provides a method for the diagnosis of disease states over a network, said method comprising the conduct of a PCCD mediated videoconference wherein the illumination of the PCCD user subject is provided by an apparatus of the present invention.

In one embodiment, the apparatus may be programmed to enable control of the illumination system by a remote videoconference participant, e.g., a healthcare worker, to provide optimal illumination of the user's body to facilitate diagnosis or provide phototherapy under remote visual control. The illumination source of the apparatus of the present invention may incorporate ultraviolet and/or infrared illumination sources to facilitate such diagnostic applications and/or to identify certain disease states, the detection of which may be enhanced using such illumination sources. The present invention further provides a method to administer phototherapy for the treatment of disease states over a network, said method comprising conduct of a PCCD mediated teleconference wherein the illumination of a the PCCD user subject is achieved using an apparatus of the present invention.

The apparatus of the present invention may be used in a variety of diagnostic applications enabling a medical professional to perform one or more diagnostic or therapeutic procedures on a subject. In one embodiment the illumination source of the apparatus may be provided to produce light in from about 360 to about 365 nm range. A lamp of this frequency is typically referred to in the field of medicine as a “Wood's Light” which is useful in the remote diagnosis of a variety of dermatological conditions. For example, a remote caregiver may activate software over a network which is communication with the control system of the apparatus of the present invention to provide remote control the illumination system of apparatus such that the illumination source of the apparatus produces ultraviolet light having a wavelength from about 360-365 nm in the direction of the patient. Using the camera of the PCCD, the remote caregiver can evaluate the patient for a variety of dermatological conditions which are highlighted by light of this frequency. For example, the apparatus of the present invention may be used in the practice of the foregoing method may be used to diagnose erythrasma caused by Corynebacterium minutissimum which fluoresces a bright coral red when illuminated with UV irradiation of the 360-365 nm range. Similarly, the apparatus of the present invention may be used in the practice of the foregoing method to diagnose Pseudomonas aeruginosa infection, especially in burns, the bacteria providing a green-yellow fluorescence when illuminated with UV irradiation of the approximately 360-365 nm range. In another application, the apparatus of the present invention may be used by a caregiver to remotely diagnose vitiligo. When the skin of an individual suffering from vitiligo is illuminated with UV illumination of the approximately 360-365 nm range, the skin fluoresces blue. The depigmentation associated with vitiligo can be differentiated from hypopigmented lesions by ivory-white color. In another application, the apparatus of the present invention may be used to diagnose acne vulgaris. In another application, the apparatus of the present invention may be used in the practice of the foregoing method to diagnose fungal infections. For example, tinea capitis caused by Microsporum canis and Microsporum audiouinii fluoresces a light bright green when illuminated with UV illumination of the approximately 360-365 nm range. The method and apparatus is also is useful to differentially diagnose tinea capitis which is caused by Trichophyton species that do not fluoresce.

Ultraviolet light photography can also be used in the diagnosis of skin cancer, particularly melanomas in conjunction with photodynamic diagnosis (PDD). In PDD, agents which produce fluorescent compounds in the presence of malignancies are used for diagnosis of cancers. For example, topical application of 5-aminolevulinic acid (ALA) has been shown to produce increased concentration of endogenous protoporphyrin IX which has high fluorescence. Studies have shown that protoporphyrin IX accumulates in skin tumors and may be used to diagnosis the presence of skin cancer. In the practice of the present invention, the patient is provided with ALA which is applied topically to a suspect lesion which may then be remotely illuminated by the apparatus of the present invention with UV light and the resulting level of fluorescence evaluated by a caregiver to diagnosis the presence of cancerous lesions. In another embodiment, the apparatus of the present invention may be used for ultraviolet light photography to define the borders of melanoma. Ultraviolet light is absorbed by melanin and the irregular pigment distribution can reveal irregular pigment distribution.

The apparatus of the present invention may be used in conjunction with other compounds in the practice of remote photodynamic diagnosis (PDD). Photodynamic diagnosis (PDD) of suspicious skin lesions relies on the fluorescent properties of an exogenous drug or endogenous compound in response to a light source. Topical agents are used to stimulate the production of endogenous photosensitizers that produce a photodynamic effect when exposed to light of certain wavelengths and energy. Topical therapies, principally used in the treatment of non-melanomatous skin cancer, such as imiquimod and 5-FU could be used in combination with remote ultraviolet photography to highlight skin cancers.

The ability of conventional digital image sensors provided with PCCD devices to detect light outside of the visible spectrum enables a variety of uses for remote medical applications. The capacity of the image sensor of the PCCD camera to detect infrared radiation enables the use of the apparatus of the present invention is useful for the remote practice of thermography, optionally in conjunction with thermostimulation (application of thermal stress to skin to be examined). Thermography is the measurement and mapping of skin surface temperature. The apparatus of the present invention enables remote monitoring of skin temperature through detection of infrared radiation by the digital image sensor of the PCCD device. The measurement of skin temperature is a useful factor in the diagnosis of a variety of conditions including inflammation and infection. Thermography has been shown to be useful in the detection and diagnosis of cutaneous melanoma and Karposi's sarcoma. Additionally, thermography can be used to detect and diagnose melanoma as melanoma lesions have been observed to be warmer than surrounding healthy tissue. Using the apparatus of the present invention, a remotely located caregiver can evaluate thermal images of skin of a remotely located patient as a non-invasive means to diagnose skin cancer. Remote thermography using the apparatus of the present invention may also be used in the remote assessment of skin burns. Differences in thermal patterns can be used to differentiate between superficial and deep skin burn wounds. Using the apparatus of the present invention, a remotely located caregiver can evaluate thermal images of skin of a remotely located patient as a non-invasive means to diagnose allergic skin conditions. Near IR wavelength (lambda=approximately 0.75 to approximately 3 micrometers) illumination can be used to evaluate water content in patients suffering from allergic dermatitis, particularly the detection and visualization of intracutaneous difference cause by the activation mechanisms of different types of allergic dermatitis. The apparatus of the present invention can also be used for infrared thermal imaging to detect signs of diabetic foot disease and to discriminate between local and diffuse diabetic foot complications.

The apparatus of the present invention can also be used for the remote evaluation of clinical samples by a medical professional. For example, a wide variety of conditions may be readily diagnosed from the observation of urine samples. In such applications the urine specimen may be placed in the proximity in the field of view of the camera and the remote caregiver may assess the properties of the clinical sample under conventional light but also under UV or IR radiation which provides extended diagnostic applications. For example, the apparatus of the present invention may be used in the practice of the foregoing method to diagnose ethylene glycol poisoning as a consequence of an individual ingesting antifreeze. Commercial antifreeze mixture typically contain fluorescein which causes the patient's urine to fluoresce when illuminated with UV irradiation of the 360-365 nm range. In another application, the method and apparatus of the present invention may be used for the remote examination and diagnosis of of porphyria cutanea tarda. Gross examination of the urine from the patient suffering from porphyria cutanea tarda is red to brown in natural light and fluoresces a pink, pink-orange to red when exposed to ultraviolet light.

Additionally, there are a wide variety of point-of-care test kits that may be used by an untrained person but require interpretation by a trained medical professional. As previously discussed, a variety of conditions may be readily diagnosed from urine specimens that are collected by a patient. Commercially available test strips such as the Multistix® and Uristix® reagent test strips (Bayer) are useful in the diagnosis of a variety of medical conditions simply by immersion into the sample. However, the interpretation of the results require a trained medical technician. In such applications, after immersion in the sample the test strip may be placed in the proximity in the field of view of the camera and the remote caregiver may assess the results.

In such configuration the apparatus of the present invention can also be used in the ensuring patient compliance with therapeutic regimens. For example, the urine of patients undergoing systemic tetracycline therapy fluoresces yellow when exposed to UV illumination. Through use of the apparatus of the present invention, the remotely located medical professional is able to ensure that the patient is complying with tetracycline therapy by evaluating the urine of the patient (as described above).

The apparatus of the present invention can also be used in ensuring patient compliance with mandatory drug testing procedures. Mandatory drug testing is frequently a condition of probation or qualification for certain governmentally administered programs. The cost of such drug testing and monitoring is significant. The ability of the apparatus of the present invention to monitor the individual through the collection process also reduces the potential for sample tampering.

The apparatus of the present invention may also be used to remotely administer phototherapy. In one embodiment, the illumination system of the apparatus provides illumination of ultraviolet wavelengths wherein the illumination control system is remotely operated by a medical technician. For example, ultraviolet B (UVB, X=approximately 280-315 nm), particularly narrow band UV (X=311-312 nm), has been demonstrated as an effective treatment for a variety of skin conditions including but not limited to acne, eczema and psoriasis. Treatment involves exposing the skin to an artificial UVB light source for a set length of time on a regular schedule. In such application, the apparatus of the present invention provides an illumination source of UVB illumination which may be administered to a remotely located individual under the control of trained personnel over a network. Alternatively, or in addition, the apparatus of the present invention may provide an illumination source providing UV-A (X=approximately 315-400 nm). In another embodiment, the phototherapy conducted using the apparatus of the present invention may be used in combination with light-sensitizing medications such as psoralen for the treatment of a variety of skin conditions such as plaque psoriasis, guttate psoriasis and psoriasis of the palms and soles. The combination of psoralen and UVA therapy (termed PUVA) may be practiced in conjunction with the apparatus of the present invention. A variety of UV illumination sources, including narrow band UV LED illumination sources, are commercially available.

The apparatus of the present invention may be used in conjunction with photodynamic therapy (PDT). Photodynamic therapy is a form of phototherapy involving the use of light and a photosensitizing chemical to elicit cell death. PDT has been shown to be useful to kill microbial cells including bacteria, fungi and viruses.

The apparatus may be provided in conjunction with software for the analysis and/or modification of the images captured through the system. In one embodiment, the software provides diagnostic analysis of the image captured using algorithms that analyze the digital image. Software applications are known in the art that are capable of through image to diagnose of certain physiological conditions (e.g. skin surface diseases such as melanoma) and provide diagnostic information to the user. For example, the images captured by the apparatus of the present invention may be evaluated and uploaded to other software programs such as Molemax (Derma Medical Systems, Handels u. Entwicklungs GmbH, Vienna, Austria).

The present invention further provides a kit of parts comprising:

1. An apparatus to provide illumination of objects for imaging by the camera of a PCCD, said apparatus comprising:

a) a housing, said housing having a means to provide stable reversible association between the housing and a PCCD,

b) an illumination system in association with said housing, said illumination system comprising at least one illumination source, said illumination system being arranged to provide illumination of objects in the proximity of said PCCD,

c) an illumination control system in operable communication with said illumination system, and

d) a power supply in electrical communication with said illumination system.

2. instructions for use.

The present invention further provides a kit of parts comprising the components 1 and 2 above and one or more components selected from the group of of power supplies, software and instructions for use. Power supplies for inclusion in the kits of the present invention including transforms, AC adapters and solar power supplies. The term software includes the inclusion of software instructions provided on computer readable media (compact disc (CD), SD, micro SD, USB cards or the like) or the provision of a “software access key” to enable downloading and/or activation of software from a remote computer system.

The invention further provides a kit of parts comprises the components 1 and 2 above further comprising at least one additional PCCD-specific insert.

### EXAMPLES

### Example 1

### Prototype

A prototype apparatus of the present invention was constructed to evaluate performance. A three dimensional housing having dimensions of 280 mm×227 mm×34 mm was employed. The front face of the housing provided a recess 242 mm×190 mm×6 mm to receive an iPad 2 Model A1395 PCCD. Retention of the PCCD was facilitated by vertical pins inserted into the lower portion of the housing and extending upward.

An illumination system consisting of 4 LED strip light fixtures (IKEA® Dioder, Article Number: 201.194.18, Inter IKEA Systems, B.V.), wired in parallel using the junction block provided, each fixture providing 9 white LEDs having a 2700K color temperature, was affixed to the face of the housing in two pairs using double faced foam adhesive tape (3M Corporation, Minneapolis, Minn.). Power supply was provided by conventional wall current with an in line IKEA model SLT5-12VS electronic transformer having a 12V DC output, 5 W max. The illumination control system was provided by a single throw in-line switch positioned between the wall plug and the transformer.

Variable illumination and color temperature was provided through the use of paper filter elements placed in front of the light strips and translucent adhesive tape. The transformer and wiring were incorporated into the housing. The housing was supported at approximately a 60 degree angle relative to the surface by a single support hingedly attached to the reverse side of the housing. During the tests, the level of ambient light was maintained at a consistent level. All tests were conducted at night to minimize inconsistencies due to varying levels of ambient sunlight.

Evaluation of the image quality was conducted by activation of the camera using the PhotoBooth® software (Apple, Inc.) and a series of photographs were taken to document the quality of the image captured by the camera with and without the illumination system and in response to various filters.

A series of PCCD mediated video csonference were initiated with three remote PCCDs: an iPad mini® (Model Number MF432LL/A), an iPhone 5C® (Model Number ME505LL/A) and an iPhone 45® (Model Number MC922LL/A) all from Apple Inc and running the FaceTime® videoconferencing software. During each video conference, the remote party was asked to evaluate the image quality. Tests were performed over a WiFi 102.11 wireless network using an iPad Model MC769LL/A) PCCD (Apple, Inc. Cupertino Calif.) having dimensions of 241.2 mm×185.7 mm×8.8 mm and possessing a 9.7 inch diagonal LED multitouch display, VGA camera, ambient light sensor and integrated microphone and speaker. The tests were conducted using the FaceTime videoconferencing software (Apple, Inc.) with and without the illumination system being activated and in response to the employment of a variety of filter elements. In each instance, the quality and clarity of the image displayed by the remote device was improved in response to the activation of the illumination system.

### Example 2

### PCCD-Specific Insert

The apparatus described in Example 1 was further modified to incorporate a PCCD-specific insert. A PCCD-specific insert was constructed having external dimensions 242 mm×190 mm×5 mm and providing a 58 mm×116 mm×5 mm recess. The iPad2 PCCD was removed from the apparatus and replaced with the PCCD-specific insert. An iPhone 4S (Model Number MC922LL/A) was inserted into the recess in the PCCD-specific insert.

A series of PCCD mediated videoconferences was conducted using the iPad 2 described above as the remote device with and without the illumination system being activated and in response to the employment of a variety of filter elements. Consistent with the results obtained from the testing described in Example 1, in each instance, the quality and clarity of the image displayed by the remote device was improved in response to the activation of the illumination system.

I claim:

1. An apparatus to provide illumination of objects for imaging by the camera of a PCCD, said apparatus comprising:
(e) a housing, said housing having a means to provide stable reversible association between the housing and a PCCD,
(f) an illumination system in association with said housing, said illumination system comprising at least one illumination source, said illumination system being arranged to provide illumination of objects in the proximity of said PCCD,
(g) an illumination control system in operable communication with said illumination system, and
(h) a power supply in electrical communication with said illumination system.

2. The apparatus of claim 1 wherein the illumination source is one or more LEDs.

3. The apparatus of claim 2 wherein the illumination control system is provided in the housing.

4. The apparatus of claim 3 wherein said power supply is a battery.

5. The apparatus of claim 2 further comprising a PCCD.

6. The apparatus of claim 5 wherein said illumination control system is provided by software of said PCCD.

7. The apparatus of claim 6 wherein the operable communication between said illumination system and said illumination control system is wireless.

8. The apparatus of claim 5 wherein the power supply is the battery of the PCCD.
The apparatus of claim 1 further comprising a PCCD-specific insert.

9. A method of providing enhanced illumination to an individual participating in a PCCD mediated videoconference said method comprising the steps of:
d) providing an apparatus to provide illumination of objects for imaging by the camera of a PCCD, said apparatus comprising:
i. a housing, said housing having a means to provide stable reversible association between the housing and a PCCD,
ii. an illumination system in association with said housing, said illumination system comprising at least one illumination source, said illumination system being arranged to provide illumination of objects in the proximity of said PCCD,
iii. an illumination control system in operable communication with said illumination system, and
iv. a power supply in electrical communication with said illumination system.
e) inserting a PCCD device into said apparatus, said PCCD device having videoconferencing software installed;
f) running the software on a PCCD device such that the camera of said PCCD is activated.

10. A method of administering telemedicine over a network to a subject in need of treatment or diagnosis, said method comprising the steps of:
a. providing the patient with an apparatus of providing comprising:
i. a housing, said housing having a means to provide stable reversible association between the housing and a PCCD,
ii. an illumination system in association with said housing, said illumination system comprising at least one illumination source, said illumination system being arranged to provide illumination of objects in the proximity of said PCCD,
iii. an illumination control system in operable communication with said illumination system, and
iv. a power supply in electrical communication with said illumination system
v. a PCCD device, said PCCD device having videoconferencing software installed,
b. activating said videoconferencing software to establish a networked teleconference communication between a caregiver and said subject,
c. activating the illumination system of said apparatus to provide illumination of the subject.

11. The method of claim 10 wherein the telemedicine procedure is a diagnostic method.

12. The method of claim 11 wherein said apparatus provides an UV illumination source providing UV illumination of said subject.

13. The method of claim 12 wherein said diagnostic method is used for the diagnosis of a condition selected from the group consisting of acne, vitiligo, erythrasma, tinea capitis, skin cancer.

14. The method of claim 11 wherein said diagnostic method is infrared thermography.

15. The method of claim 14 wherein said diagnostic method is used for the diagnosis of condition selected from the group consisting of melanoma, burns, allergies, and diabetic foot diease.

15. The method of claim 10 wherein said telemedicine procedure is remote evaluation of clinical samples.

16. The method of claim 10 wherein said telemedicine procedure is phototherapy.

17. The method of claim 16 wherein said phototherapy procedure is used for the treatment of a condition selected from the group consisting of acne, eczema and psoriasis.

18. The method of claim 16 wherein said phototherapy is photodynamic therapy.
